(19) United States
(12) Patent Application Publication
(10) Pub. No.: US 2022/0298131 A1
(43) Pub. Date: Sep. 22, 2022

(54) METHOD OF MAKING GLYCEROL CARBONATE (METH)ACRYLATE AND CURABLE COMPOSITIONS BASED THEREON

(71) Applicant: Arkema France, Colombes (FR)

(72) Inventors: Sean NUNEZ, Exton, PA (US); Kyle SNOW, Exton, PA (US)

(21) Appl. No.: 17/630,965

(22) PCT Filed: Jul. 27, 2020
(86) PCT No.: PCT/IB2020/000652

§ 371 Date: Jan. 28, 2022

Related U.S. Application Data
(60) Provisional application No. 62/880,864, filed on Jul. 31, 2019.

(51) Int. Cl. C07D 317/36

(52) U.S. Cl. CPC C07D 317/36; C08F 220/14

(57) ABSTRACT

Glycerol carbonate methacrylate may be prepared by reacting glycerol monomethacrylate and a carbonate such as a dialkyl carbonate in the presence of a catalyst and may be combined with an actinic radiation-curable oligomer, such as a (meth)acrylate-functionalized oligomer, and possibly other components to provide compositions capable of being cured, for example by exposure to actinic radiation, to obtain polymeric materials, such as 3D printed articles. The glycerol carbonate methacrylate reduces the viscosity of the composition, which may in the absence of the glycerol carbonate methacrylate be too high for the curable composition to be readily processed at room temperature, but additionally can impart a variety of other useful attributes to the curable compositions and cured products derived therefrom.

### FIELD OF THE INVENTION

The present invention relates to processes for making glycerol carbonate (meth)acrylate, as well as to curable compositions containing glycerol carbonate (meth)acrylate.

### BACKGROUND OF THE INVENTION

Glycerol carbonate methacrylate (i.e., the methacrylic acid ester of glycerol carbonate, which is also referred to as glycerin carbonate methacrylate) has been identified as a useful synthetic intermediate and monomer, having the following structure:

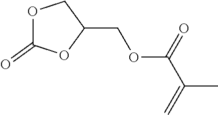

Japanese Patent Application Laid-Open No. 2011-219394, for example, teaches that (meth)acrylic acid esters having a 2-oxo-1,3-dioxolane (cyclic carbonate) structure, such as glycerol carbonate methacrylate, can be used as a raw material for paints, functional polymers, raw materials for medicines, agricultural chemicals and other fine chemicals.

Although different synthetic routes to glycerol carbonate methacrylate have been described in the literature, the processes of most interest involve the reaction of glycidyl methacrylate with carbon dioxide in the presence of a suitable catalyst. Such chemistry is described, for example, in U.S. Pat. No. 4,835,289 and Japanese Patent Application Laid-Open No. 2014-051456. While glycerol carbonate methacrylate can be obtained in good yield using such a process, one disadvantage of this synthetic route is that glycidyl methacrylate must be employed as a starting material. Glycidyl methacrylate is generally prepared commercially by reacting epichlorohydrin with methacrylic acid. The resulting reaction product typically is contaminated with unreacted epichlorohydrin, which is corrosive and recognized as having significant health and safety concerns. If not removed, the residual epichlorohydrin in the glycerol carbonate methacrylate can interfere with the ability to formulate the glycerol carbonate methacrylate into various compositions such as coatings, inks, 3D printed articles and the like that may come into contact with human skin or other biological living tissues. Additionally, the regulatory classification of such compositions and products may be affected by the presence of residual epichlorohydrin. Accordingly, the development of viable alternative methods for synthesizing glycerol carbonate methacrylate that do not involve the use of epichlorohydrin-containing starting materials would be of significant interest.

Curable compositions containing glycerol carbonate methacrylate as a component have, to date, received little attention. Camara et al., European Polymer Journal 61 (2014) 133-144, reported what was said to be the first complete study of the free radical polymerization of glycerol carbonate methacrylate to synthesize cyclic carbonate-functionalized polymers, including copolymers with methyl methacrylate and other acrylic, methacrylic and styrenic monomers. Example 9 of U.S. Pat. Application Publication No. 2017/0260418 A1 describes an ink, which is used as Part A of a two part ink for 3D printing and which contains glycerol carbonate methacrylate, triethylene glycol dimethacrylate, and a photoinitiator. However, according to the published patent application, such an ink must be combined with an amine monomer-containing ink (“Part B”) to render it suitable for use in 3D printing, wherein the amine monomer contains one or more primary, secondary and/or tertiary amine groups.

EP 0001088 A1 discloses polymers containing 1,3-dioxalan-2-one groups in the side chain which are obtained by polymerization of the corresponding unsaturated compound, such as glycerol carbonate methacrylate. Co-polymerization with other olefinically unsaturated monomers is also described. The polymers can be used to produce moldings or molding compounds, coatings, adhesives and paper and textile auxiliaries. There is no mention of achieving such polymerization by means of photocuring, nor does the publication disclose co-polymerizing carbonate-containing monomers such as glycerol carbonate methacrylate with olefinically unsaturated oligomers.

U.S. Pat. No. 5,047,261 discloses a process for the manufacture of coatings by radio-crosslinking a radio-crosslinkable composition having a reactive diluent system containing at least one mono(meth)acrylic carbonate corresponding to a particular formula. Glycerol carbonate acrylate was used as a monomer in comparative Examples 9 and 21, but the patent does not disclose radio-crosslinkable compositions containing glycerol carbonate methacrylate.

Generally speaking, methacrylate compounds (i.e., compounds containing one or more methacrylate functional groups, —OC(═O)C(CH₃)═CH₂) are recognized as being much slower to react and cure when exposed to actinic radiation than the analogous acrylate compounds (i.e., compounds containing one or more acrylate functional groups, —OC(═O)CH═CH₂).

### SUMMARY OF THE INVENTION

The present inventors have now discovered that glycerol carbonate methacrylate can be readily prepared in high yield by reacting glycerol monomethacrylate with a carbonate selected from the group consisting of dialkyl carbonates and cyclic alkylene carbonates in the presence of a catalyst. Surprisingly, the methacrylate functional group substantially survives such reaction, wherein a cyclic carbonate group is formed by interchange of the carbonate reactant with the hydroxyl groups of the glycerol monomethacrylate. An alcohol co-product is produced together with the glycerol carbonate methacrylate, but can be readily separated by distillation or other such means. The transformation of the starting glycerol monomethacrylate into the desired glycerol carbonate methacrylate may be schematically represented as follows:

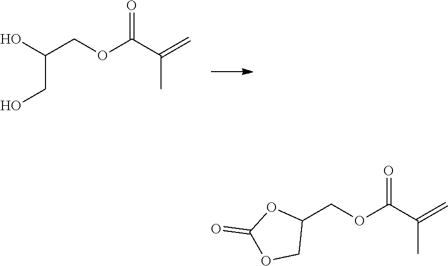

Other types of co-reactants may be substituted for the carbonate, in particular co-reactants which are capable of functioning as synthetic equivalents of a dialkyl carbonate or cyclic alkylene carbonate. Such alternative co-reactants include compounds comprising a carbonyl group in which the carbon atom of the carbonyl group is substituted with two groups capable of being displaced, in effect, by the hydroxyl groups of the glycerol monomethacrylate. Such substituent groups may, for example, be aroxy (e.g., phenoxy), alkoxy (including halogenated alkoxy, such as Cl₃CO—), halo (e.g., Cl, Br), alkylthio or amino groups. Suitable alternative co-reactants include, for example, compounds corresponding to the general formula XC(═O)Y wherein X and Y are the same as or different from each other and are selected from the group consisting of aroxy, alkoxy, halo, alkylthio (e.g., RS—, wherein R is an alkyl group) and amino (including —NH₂, —NHR and —NR₂, wherein R is an organic group and the nitrogen atom may be part of a ring structure, such as in an imidazole or benzotriazole group). X and Y may be linked together to form a cyclic structure. Examples of suitable non-carbonate co-reactants include, but are not limited to, phosgene, triphosgene, urea, carbonyldiimidazole, carbonyldibenzotriazoles, dimethyldithiocarbonates, phenyl chloroformates, trihaloacetyl chlorides, and nitrophenyl benzylcarbamates.

The starting material glycerol monomethacrylate (also known as 2,3-dihydroxypropyl methacrylate) may be prepared by any known method, such as the monoesterification of glycerol with a methacrylate source such as methacrylic acid, methacrylic anhydride, methacryloyl chloride or lower alkyl ester of methacrylic acid or the hydrolysis of the epoxy group in glycidyl methacrylate. Other methods are described, for example, in U.S. Pat. No. 7,342,054 B2, WO 00/63149, and WO 00/63150. One advantage of the present inventive process for preparing glycerol carbonate methacrylate is that the starting material glycerol monomethacrylate is not prepared using epichlorohydrin. For example, an epichlorohydrin-free grade of glycidyl methacrylate may be used as a precursor for the glycerol monomethacrylate. Thus, the preparation of glycerol carbonate methacrylate which is epichlorohydrin-free is feasible, in contrast to the known synthetic routes which utilize glycidyl methacrylate as a starting material.

Additionally, the inventors have established that compositions capable of being readily cured by exposure to actinic radiation to form useful polymeric products may be formulated using glycerol carbonate methacrylate in combination with one or more actinic radiation-curable oligomers (in particular, one or more (meth)acrylate-functionalized oligomers), together with possibly one or more other components such as photoinitiators and/or actinic radiation-curable monomers (such as (meth)acrylate-functionalized monomers) in addition to the glycerol carbonate methacrylate. The properties of glycerol carbonate methacrylate make it particularly well-suited for use in such applications. Glycerol carbonate methacrylate has a low viscosity at ambient temperatures (55-65 cps at 25° C.) and thus is capable of functioning as a reactive diluent, thereby effectively reducing the viscosity of curable compositions containing high proportions of actinic radiation-curable oligomers. When homopolymerized, glycerol carbonate methacrylate yields a homopolymer having a high glass transition temperature (>160° C.), high tensile strength (>18 MPa), and high tensile modulus (80 MPa). Accordingly, the incorporation of glycerol carbonate methacrylate into an actinic radiation-curable oligomer-containing curable composition serves to significantly improve the physical and mechanical properties of a cured polymeric matrix obtained therefrom. Furthermore, glycerol carbonate methacrylate has no acute toxicities, in contrast to other (meth)acrylate-functionalized compounds which could also be used as reactive diluents. Accordingly, articles may be prepared from curable compositions in accordance with the present invention which are suitable for use in medical device and medical use applications in which such articles are brought into contact with a human subject.

Additionally, glycerol carbonate methacrylate displays polymerization kinetics which are atypical of methacrylate-functionalized compounds. Acrylates and methacrylates generally have different reactivities when polymerized using actinic radiation, with methacrylates curing significantly more slowly than the corresponding acrylates. As will be explained in more detail subsequently, the present inventors have found that glycerol carbonate methacrylate can be used in methacrylate-based formulations to increase radiation cure speed and flexural strength (green strength).

### DESCRIPTION OF THE DRAWINGS

FIGS. 1-8 depict various types of experimental data, as explained in the Examples.

### DETAILED DESCRIPTION OF EMBODIMENTS OF THE INVENTION

### Synthesis of Glycerol Carbonate Methacrylate from Glycerol Monomethacrylate and a Carbonate

Suitable carbonates for reacting with the glycerol monomethacrylate include carbonates selected from the group consisting of dialkyl carbonates and cyclic alkylene carbonates. Although mixtures of such carbonates could be used, in certain embodiments only a single carbonate co-reactant is employed. Suitable dialkyl carbonates include in particular carbonates in which the alkyl groups are lower alkyl groups such as, for example, C1-C6 alkyl groups, which may be straight chain or branched. For example, the alkyl groups may be methyl, ethyl, propyl (including n-propyl and isopropyl), and butyl (including n-butyl, sec-butyl and tert-butyl). Suitable cyclic alkylene carbonates include, by way of example, ethylene carbonate and propylene carbonate. According to certain desirable embodiments of the invention, the carbonate is selected such that the alcohol co-product(s) generated have a boiling point at atmospheric pressure of 200° C. or less, 175° C. or less, 150° C. or less, 125° C. or less, or 100° C. or less, to facilitate separation of the co-product alcohol formed, either during or after reaction of the glycerol monomethacrylate and the carbonate.

Any suitable molar ratio of carbonate to glycerol monomethacrylate which favors the formation of glycerol carbonate methacrylate can be used for reaction. However, at least stoichiometric levels of carbonate relative to glycerol monomethacrylate are typically used, with a moderate molar excess of the carbonate generally being preferred. Preferably, the molar ratio of carbonate to glycerol monomethacrylate is in the range of 1:1 to 3:1. For example, in one embodiment the carbonate : glycerol monomethacrylate molar ratio is 1.1:1 to 1.2:1.

Suitable catalysts include any substances capable of accelerating the rate of reaction between the glycerol monomethacrylate and the carbonate, including Lewis acids, Lewis bases, Bronsted bases, and basic catalysts (Lewis or Bronsted) generally. The catalyst may be homogeneous (dissolved in the reaction mixture under the reaction conditions) or heterogeneous (undissolved in the reaction mixture under the reaction conditions). It is also possible for the catalyst to be partially dissolved under the reaction conditions. Mixtures of two or more different catalysts may be used.

Suitable basic catalysts include, without limitation, alkali metal (e.g., Li, Na, K) and alkaline earth metal (e.g., Ca, Mg) compounds, which may be organic (i.e., containing one or more organic moieties in addition to the alkali metal and/or alkaline earth metal) or inorganic in nature. Such compounds may be, for example, hydroxides, alkoxides, carbonates, bicarbonates, silicates, aluminates, oxides and the like. Basic ion exchange resins or basic zeolites could also be employed. According to certain embodiments of the invention, the catalyst is a strong base, i.e., a base having a pKb value of at most 5.

Suitable catalysts include basic catalysts selected from alkali metal carbonates, alkali metal bicarbonates, alkali metal hydroxides, alkali metal oxides, alkali metal alkoxides, alkali metal aluminates, alkali metal silicates, alkaline earth metal carbonates, alkaline earth metal bicarbonates, alkaline earth metal hydroxides, alkaline earth metal oxides, alkaline earth metal alkoxides, alkaline earth metal aluminates, alkaline earth metal silicates and combinations thereof. In preferred embodiments, the catalyst is an alkali metal hydroxide or alkali metal alkoxide. Reference to “alkoxide” herein includes C₁ to C₆ straight chain or branched aikoxides, for example C₁ to C₂ alkoxides. Specific examples of suitable catalysts include NaOH, KOH, NaOMe, NaOEt, KOMe, KOEt, Na₂CO₃, Na CO₃, K₂CO₃, KHCO₃, and Na₂SiO₃. Amine compounds, including tertiary amines, may also be utilized as suitable catalysts. Also suitable for use as catalysts are the substances known as “ionic liquids,” such as those described in WO 2017/125759 (incorporated herein by reference in its entirety for all purposes). Exemplary ionic liquids for this purpose include ionic liquids containing ammonium or phosphonium cations or aromatic heterocyclic cationic species. The catalyst may be combined with the reactants in dry or neat form, but in certain embodiments may be provided in solution or slurry form in combination with a solvent.

The catalyst is supplied to the initially formed reaction mixture in an amount effective to achieve the desired catalytic effect. In certain embodiments, the catalyst is present in the reaction mixture in an amount of from 0.05 to 5% by weight, based on the weight of the entire initially formed reaction mixture.

One or more polymerization inhibitors (in particular, free radical inhibitors) may be present in the reaction mixture to help reduce undesired reactions of the (meth)acrylate functionality. Suitable polymerization inhibitors include, for example, hydroquinone polymerization inhibitors (e.g., hydroquinone itself as well as substituted hydroquinones such as hydroquinone monomethyl ether); hindered phenolic polymerization inhibitors (such as butylated hydroxytoluene); and thiazine polymerization inhibitors (such as phenothiazine). The level of polymerization inhibitor in the reaction mixture may be varied depending upon the type of inhibitor used and other factors, but typically may be from about 5 to about 10,000 ppm.

Polymerization of the (meth)acrylate-functionalized components of the reactions mixture may also be suppressed by carrying out the reaction of the glycerol mono(meth)acrylate and carbonate in the presence of molecular oxygen. For example, the reaction mixture may be sparged with a gas (such as air) which is comprised of molecular oxygen, The gas fed to a reaction vessel in which the reaction is being conducted may, for instance, atmospheric air, enriched air, or air in which the molecular oxygen content has been reduced from normal (atmospheric) levels in order to reduce the potential for flammability.

Although one or more solvents could be used in the process of the present invention, such a solvent is not required. Thus, in one embodiment of the invention, the reaction between the glycerol monomethacrylate and the carbonate is conducted in the absence of solvent. For instance, the reaction mixture (as initially formed) may contain less than 500 ppm or less than 200 ppm solvent.

The components of the reaction mixture (glycerol monomethacrylate, carbonate, catalyst, optional solvent and possibly other optional components) may be charged to a suitable reaction vessel, either all at once or sequentially. For example, the carbonate may be added in two or more portions to a mixture of the other components of the reaction mixture. The components may be stirred, mechanically mixed or otherwise agitated while conducting the desired reaction involving the glycerol monomethacrylate and the carbonate.

The glycerol monomethacrylate and carbonate are reacted in the presence of the catalyst for a time and at a temperature and pressure effective to form the desired glycerol carbonate methacrylate in the target yield and selectivity. Generally speaking, the temperature is selected to be a temperature or range of temperatures at which the reaction takes place at a

commercially practical rate while avoiding, minimizing or reducing decomposition of the reactants or formation of undesired byproducts and polymers. Suitable reaction temperatures include, for example, 40° C. to 160° C.

Suitable reaction times may, be on the order of from a few minutes (e.g., at least 10 minutes) up to several hours (e.g., up to 12 hours).

In order to help drive the desired reaction to form the glycerol carbonate monomethacrylate closer or more quickly to completion, it may be helpful to separate the co-product alcohol(s) formed during the reaction from the reaction mixture. As will be appreciated by the skilled person, the term “separate” is intended to refer to the physical extraction of alcohol co-product from the reaction mixture. An alcohol-containing co-product strewn may be obtained as a result. Such separation may be carried out on a continuous basis or in stages. For example, the reaction may be conducted for a defined period of time without removing any of the co-product alcohol before subjecting the reaction mixture to a separation procedure (such as flash distillation or column distillation), before continuing to react the components of the reaction mixture in a further reaction stage. The recovered alcohol co-product may be recycled (i.e., converted back into a carbonate to be used again to prepare glycerol carbonate (meth)acrylate) or utilized in some other capacity.

When the desired degree of conversion of one or more of the glycerol monomethacrylate or carbonate is achieved, reaction may be discontinued and the reaction product comprising the target glycerol carbonate methacrylate thereafter subjected to a suitable work-up or purification procedure to obtain the glycerol carbonate methacrylate in the desired state of purity. Such purification steps may include, for example, washing and/or neutralization to remove or deactivate the catalyst, drying, treatment with an adsorbent, decolorization and/or fractional distillation.

### Curable Compositions Containing Glycerol Carbonate Methacrylate and Actinic Radiation-Curable Oligomer

One aspect of the present invention provides a curable composition which is comprised of glycerol carbonate methacrylate and at least one actinic radiation-curable oligomer (such as at least one (meth)acrylate-functionalized oligomer). Such compositions may be photocurable or radiation-curable, i.e., capable of being cured by exposure to actinic radiation such as UV light, visible light or electron beam radiation. The glycerol carbonate methacrylate may function as a reactive diluent and reduce the viscosity of the at least one actinic radiation-curable oligomer; such oligomers, particularly if relatively high in molecular weight, tend to have high viscosities or may even be solid in neat form at ambient temperatures (e.g., 25° C.). The glycerol carbonate methacrylate may render the curable composition sufficiently low in viscosity, even without solvent being present, that the curable composition can be easily applied at a suitable application temperature to a substrate surface so as to form a relatively thin, uniform layer. Thus, according to certain embodiments, the at least one actinic radiation-curable oligomer may have a viscosity at 25° C. in neat form of at least 10,000 cPS and the glycerol carbonate methacrylate is present in the curable composition an amount effective to provide the curable composition with a viscosity at 25° C. of less than 10,000 cPs (preferably less than 2500 cPs).

The amount of glycerol carbonate methacrylate in the curable composition may be varied as may be desired depending upon the properties desired in both the curable composition and cured products obtained therefrom. For example, and without limitation, the curable composition may comprise at least 1%, at least 2%, at least 5%, at least 10%, at least 15%, at least 20%, or at least 25% by weight, in total, of glycerol carbonate methacrylate, based on the total weight of the curable composition. The maximum amount of glycerol carbonate methacrylate is not necessarily limited, keeping in mind that the curable compositions of the present invention additionally contain at least some amount of actinic radiation-curable oligomer and possibly other components as well (e.g., photoinitiator and/or reactive substances other than actinic radiation-curable oligomer and glycerol carbonate methacrylate, such as one or more actinic radiation-curable monomers). For instance, the curable composition could comprise up to 95%, up to 90%, up to 85%, up to 80%, or up to 75% by weight, in total, of glycerol carbonate methacrylate, based on the total weight of the curable composition. The content of glycerol carbonate methacrylate will vary depending upon the end-use application, but typically will be from 10 to 65% by weight based on the total weight of the curable composition. According to certain embodiments, the curable composition is comprised of 20 to 30% by weight glycerol carbonate methacrylate based on the total weight of the curable composition.

### Actinic Radiation-Curable Oligomer

The amount of actinic radiation-curable oligomer (e.g., (meth)acrylate-functionalized oligomer) may be varied as may be desired depending upon the type or types of oligomers used as well as the properties desired in both the curable composition and cured products obtained therefrom. For example, and without limitation, the curable composition may comprise at least 1%, at least 2%, at least 5%, at least 10%, at least 15%, at least 20%, or at least 25% by weight, in total, of actinic radiation-curable oligomer (e.g., (meth)acrylate-functionalized oligomer), based on the total weight of the curable composition. The maximum amount of actinic radiation-curable oligomer (e.g., (meth)acrylate-functionalized oligomer) is not necessarily limited, keeping in mind that the curable compositions of the present invention additionally contain at least some amount of glycerol carbonate methacrylate and possibly other components as well (e.g., photoinitiator and/or reactive substances other than actinic radiation-curable oligomer and glycerol carbonate methacrylate, such as one or more actinic radiation-curable monomers). For instance, the curable composition could comprise up to 95%, up to 90%, up to 85%, up to 80%, or up to 75% by weight, in total, of actinic radiation-curable curable oligomer (e.g., (meth)acrylate-functionalized oligomer), based on the total weight of the curable composition. The content of oligomer will vary depending upon the end-use application, but typically will be from 10 to 65% by weight based on the total weight of the curable composition. According to certain embodiments, the curable composition is comprised of 20 to 30% by weight oligomer based on the total weight of the curable composition.

The types of actinic radiation-curable oligomers which may be utilized in combination with glycerol carbonate methacrylate to produce curable compositions in accordance with the present invention are not particularly limited and any of such oligomers known in the art can be employed. Actinic radiation-curable oligomers include any oligomeric substances containing at least one functional group per molecule capable of being cured (reacted) when exposed to actinic radiation. Such actinic radiation-curable functional groups include functional groups containing sites of ethylenic unsaturation (i.e., carbon-carbon double bonds, C═C) such as acrylate (including cyanoacrylate), methacrylate, acrylamide, methacrylamide, maleyl, allyl, propenyl and vinyl functional groups and combinations thereof. The use of (meth)acrylate-functionalized oligomers can be particularly advantageous. Especially suitable for such purpose are (meth)acrylate-functionalized oligomers selected from the group consisting of (meth)acrylate-functionalized urethane oligomers (sometimes also referred to as “urethane (meth)acrylate oligomers,” “polyurethane (meth)acrylate oligomers” or “carbamate (meth)acrylate oligomers”), (meth)acrylate-functionalized epoxy oligomers (sometimes also referred to as “epoxy (meth)acrylate oligomers”), (meth)acrylate-functionalized polyether oligomers (sometimes also referred to as “polyester (meth)acrylate oligomers”), (meth)acrylate-functionalized polydiene oligomers (sometimes also referred to as “polydiene (meth)acrylate oligomers”), (meth)acrylate-functionalized polycarbonate oligomers (sometimes also referred to as “polycarbonate (meth)acrylate oligomers”), and (meth)acrylate-functionalized polyester oligomers (sometimes also referred to as “polyester (meth)acrylate oligomers”). According to certain embodiments, at least one of the oligomers is a methacrylate-functionalized oligomer. In other embodiments, all of the oligomers present in the curable composition are methacrylate-functionalized oligomers.

According to certain aspects of the invention, the curable composition, when subjected to curing to form a polymeric matrix, does not contain any amino-containing compound (oligomeric or monomeric), wherein “amino” as used herein refers to a primary, secondary or tertiary amine group, but does not include any other type of nitrogen-containing group such as an amide, carbamate (urethane), urea, or sulfonamide group. Thus, the curable composition may be employed in the form of a one-part system that is exposed to actinic radiation and cured without being combined with an amino-containing compound having amino groups which interact chemically with the glycerol carbonate methacrylate as part of the curing process.

A (meth)acrylate-functionalized oligomer may be generally defined as an organic substance which is oligomeric in character and which contains at least one acrylate or methacrylate functional group per molecule.

Any of the (meth)acrylate-functionalized oligomers known in the art may be used in the curable compositions of the present invention. According to certain embodiments, such oligomers may contain two or more (meth)acrylate functional groups per molecule. The number average molecular weight of such oligomers may vary widely, e.g., from about 500 to about 50,000 daltons. Such oligomers may be selected and used in combination with the glycerol carbonate methacrylate and optionally one or more (meth)acrylate-functionalized monomers other than glycerol carbonate methacrylate in order to enhance the flexibility, strength and/or modulus, among other attributes, of a cured polymer prepared using the curable composition of the present invention.

Exemplary polyester (meth)acrylate oligomers include the reaction products of acrylic or methacrylic acid or mixtures or synthetic equivalents thereof with hydroxyl group-terminated polyester polyols. The reaction process may be conducted such that all or essentially all of the hydroxyl groups of the polyester polyol have been (meth)acrylated, particularly in cases where the polyester polyol is difunctional. The polyester polyols can be made by polycondensation reactions of polyhydroxyl functional components (in particular, diols) and polycarboxylic acid functional compounds (in particular, dicarboxylic acids and anhydrides). The polyhydroxyl functional and polycarboxylic acid functional components can each have linear, branched, cycloaliphatic or aromatic structures and can be used individually or as mixtures.

Examples of suitable epoxy (meth)acrylate oligomers include the reaction products of acrylic or methacrylic acid or mixtures thereof with glycidyl ethers or esters, such as glycidyl ethers of bis-phenol compounds and oligomers thereof.

Suitable polyether (meth)acrylate oligomers include, but are not limited to, the condensation reaction products of acrylic or methacrylic acid or synthetic equivalents or mixtures thereof with polyetherols which are polyether polyols (such as polyethylene glycol, polypropylene glycol or polytetramethylene glycol). Suitable polyetherols can be linear or branched substances containing ether bonds and terminal hydroxyl groups. Polyetherols can be prepared by ring opening polymerization of cyclic ethers such as tetrahydrofuran or alkylene oxides (e.g., ethylene oxide and/or propylene oxide) with a starter molecule. Suitable starter molecules include water, polyhydroxyl functional materials, polyester polyols and amines.

Polyurethane (meth)acrylate oligomers (sometimes also referred to as “urethane (meth)acrylate oligomers”) capable of being used in the curable compositions of the present invention include urethanes based on aliphatic and/or aromatic polyester polyols and polyether polyols and aliphatic and/or aromatic polyester diisocyanates and polyether diisocyanates capped with (meth)acrylate end-groups. Suitable polyurethane (meth)acrylate oligomers include, for example, aliphatic polyester-based urethane di- and tetra-acrylate oligomers, aliphatic polyether-based urethane di- and tetra-acrylate oligomers, as well as aliphatic polyester/polyether-based urethane di- and tetra-acrylate oligomers.

In various embodiments, the polyurethane (meth)acrylate oligomers may be prepared by reacting aliphatic and/or aromatic diisocyanates with OH group terminated polyester polyols (including aromatic, aliphatic and mixed aliphatic/aromatic polyester polyols), polyether polyols, polycarbonate polyols, polycaprolactone polyols, polyorganosiloxane polyols (e.g., polydimethylsiloxane polyols), or polydiene polyols (e.g., polybutadiene polyols), or combinations thereof to form isocyanate-functionalized oligomers which are then reacted with hydroxyl-functionalized (meth)acrylates such as hydroxyethyl acrylate or hydroxyethyl methacrylate to provide terminal (meth)acrylate groups. For example, the polyurethane (meth)acrylate oligomers may contain two, three, four or more (meth)acrylate functional groups per molecule. Alternative synthetic approaches may also be used to prepare suitable (meth)acrylate-functionalized urethane oligomers, such as by reacting any of the aforementioned polyols with isocyanate-functionalized (meth)acrylates (e.g., the 1:1 reaction product of a diisocyanate and a hydroxyalkyl (meth)acrylate).

Suitable acrylic (meth)acrylate oligomers (sometimes also referred to in the art as “acrylic oligomers”) include oligomers which may be described as substances having an oligomeric acrylic backbone which is functionalized with one or (meth)acrylate groups (which may be at a terminus of the oligomer or pendant to the acrylic backbone). The acrylic backbone may be a homopolymer, random copolymer or block copolymer comprised of repeating units of acrylic monomers. The acrylic monomers may be any monomeric (meth)acrylate such as C1-C6 alkyl (meth)acrylates as well as functionalized (meth)acrylates such as (meth)acrylates bearing hydroxyl, carboxylic acid and/or epoxy groups. Acrylic (meth)acrylate oligomers may be prepared using any procedures known in the art, such as by oligomerizing monomers, at least a portion of which are functionalized with hydroxyl, carboxylic acid and/or epoxy groups (e.g., hydroxyalkyl(meth)acrylates, (meth)acrylic acid, glycidyl (meth)acrylate) to obtain a functionalized oligomer intermediate, which is then reacted with one or more (meth)acrylate-containing reactants to introduce the desired (meth)acrylate functional groups.

### Actinic Radiation-Curable Monomer

The curable compositions may additionally comprise at least one actinic radiation-curable monomer (such as a (meth)acrylate-functionalized monomer) other than glycerol carbonate methacrylate. Actinic radiation-curable monomers include any monomeric (non-oligomeric) substances containing at least one functional group per molecule capable of being cured (reacted) when exposed to actinic radiation. Such actinic radiation-curable functional groups include functional groups containing sites of ethylenic unsaturation (i.e., carbon-carbon double bonds, C═C) such as acrylate (including cyanoacrylate), methacrylate, acrylamide, methacrylamide, maleyl, allyl, propenyl and vinyl functional groups and combinations thereof. The use of (meth)acrylate-functionalized monomers is particularly advantageous.

For example, according to certain embodiments, the curable composition is additionally comprised of at least one methacrylate-functionalized monomer other than glycerol carbonate methacrylate. However, in other embodiments, the curable composition may comprise one or more acrylate-functionalized monomers and one or more methacrylate-functionalized monomers.

A (meth)acrylate-functionalized monomer may be generally defined as an organic substance which is non-oligomeric in character and which contains at least one acrylate or methacrylate functional group per molecule. According to certain aspects of the invention, the (meth)acrylate-functionalized monomer(s) used may be relatively low in molecular weight (e.g., a number average molecular weight of 100 to 1000 daltons).

The curable composition of the present invention may comprise, for example, at least one (meth)acrylate-functionalized monomer containing two or more (meth)acrylate functional groups per molecule. Examples of useful (meth)acrylate-functionalized monomers containing two or more (meth)acrylate functional groups per molecule include acrylate and methacrylate esters of polyhydric alcohols (organic compounds containing two or more, e.g., 2 to 6, hydroxyl groups per molecule). Specific examples of suitable polyhydric alcohols include C₂₋₂₀ alkylene glycols (glycols having a C₂₋₁₀ alkylene group may be preferred, in which the carbon chain may be branched; e.g., ethylene glycol, trimethylene glycol, 1,2-propylene glycol, 1,2-butanediol, 1,3-butanediol, 2,3-butanediol, tetramethylene glycol (1,4-butanediol), 1,5-pentanediol, 1,6-hexanediol, 1,8-octanediol, 1,9-nonanediol, 1,12-dodecanediol, cyclohexane-1,4-dimethanol, bisphenols, and hydrogenated bisphenols, as well as alkoxylated (e.g., ethoxylated and/or propoxylated) derivatives thereof), diethylene glycol, glycerin, alkoxylated glycerin, triethylene glycol, dipropylene glycol, tripropylene glycol, trimethylolpropane, alkoxylated trimethylolpropane, ditrimethylolpropane, alkoxylated ditrimethylolpropane, pentaerythritol, alkoxylated pentaerythritol, dipentaerythritol, alkoxylated dipentaerythritol, cyclohexanediol, alkoxylated cyclohexanediol, cyclohexanedimethanol, alkoxylated cyclohexanedimethanol, norbornene dimethanol, alkoxylated norbornene dimethanol, norbornane dimethanol, alkoxylated norbornane dimethanol, polyols containing an aromatic ring, cyclohexane-1,4-dimethanol ethylene oxide adducts, bis-phenol ethylene oxide adducts, hydrogenated bisphenol ethylene oxide adducts, bisphenol propylene oxide adducts, hydrogenated bisphenol propylene oxide adducts, cyclohexane-1,4-dimethanol propylene oxide adducts, sugar alcohols and alkoxylated sugar alcohols. Such polyhydric alcohols may be fully or partially esterified (with (meth)acrylic acid, (meth)acrylic anhydride, (meth)acryloyl chloride or the like), provided they contain at least two (meth)acrylate functional groups per molecule. As used herein, the term “alkoxylated” refers to compounds containing one or more oxyalkylene moieties (e.g., oxyethylene and/or oxypropylene moieties). An oxyalkylene moiety corresponds to the general structure —R—O—, wherein R is a divalent aliphatic moiety such as —CH₂CH₂— or —CH₂CH(CH₃)—. For example, an alkoxylated compound may contain from 1 to 25 oxyalkylene moieties per molecule.

Exemplary (meth)acrylate-functionalized monomers containing two or more (meth)acrylate functional groups per molecule may include ethoxylated bisphenol A di(meth)acrylates; triethylene glycol di(meth)acrylate; ethylene glycol di(meth)acrylate; tetraethylene glycol di(meth)acrylate; polyethylene glycol di(meth)acrylates; 1,4-butanediol diacrylate; 1,4-butanediol dimethacrylate; diethylene glycol diacrylate; diethylene glycol dimethacrylate, 1,6-hexanediol diacrylate; 1,6-hexanediol dimethacrylate; neopentyl glycol diacrylate; neopentyl glycol di(meth)acrylate; polyethylene glycol (600) dimethacrylate (where 600 refers to the approximate number average molecular weight of the polyethylene glycol portion); polyethylene glycol (200) diacrylate; 1,12-dodecanediol dimethacrylate;

tetraethylene glycol diacrylate; triethylene glycol diacrylate, 1,3-butylene glycol dimethacrylate, tripropylene glycol diacrylate, polybutadiene diacrylate; methyl pentanediol diacrylate; polyethylene glycol (400) diacrylate; ethoxylated₂ bisphenol A dimethacrylate; ethoxylated₃ bisphenol A dimethacrylate; ethoxylated₃ bisphenol A diacrylate; cyclohexane dimethanol dimethacrylate; cyclohexane dimethanol diacrylate; ethoxylated₁₀ bisphenol A dimethacrylate (where the numeral following “ethoxylated” is the average number of oxyalkylene moieties per molecule); dipropylene glycol diacrylate; ethoxylated₄ bisphenol A dimethacrylate; ethoxylated₆ bisphenol A dimethacrylate; ethoxylated₈ bisphenol A dimethacrylate; alkoxylated hexanediol diacrylates; alkoxylated cyclohexane dimethanol diacrylate; dodecane diacrylate; ethoxylated₄ bisphenol A diacrylate; ethoxylated₁₀ bisphenol A diacrylate; polyethylene glycol (400) dimethacrylate; polypropylene glycol (400) dimethacrylate; metallic diacrylates; modified metallic diacrylates; metallic dimethacrylates; polyethylene glycol (1000) dimethacrylate; methacrylated polybutadiene; propoxylated₂ neopentyl glycol diacrylate; ethoxylated₃₀ bisphenol A dimethacrylate; ethoxylated₃₀ bisphenol A diacrylate; alkoxylated neopentyl glycol diacrylates; polyethylene glycol dimethacrylates; 1,3-butylene glycol diacrylate; ethoxylated₂ bisphenol A dimethacrylate; dipropylene glycol diacrylate; ethoxylated₄ bisphenol A diacrylate; polyethylene glycol (600) diacrylate; polyethylene glycol (1000) dimethacrylate; tricyclodecane dimethanol diacrylate; propoxylated neopentyl glycol diacrylates such as propoxylated₂ neopentyl glycol diacrylate; diacrylates of alkoxylated aliphatic alcohols; trimethylolpropane trimethacrylate; trimethylolpropane triacrylate; tris (2-hydroxyethyl) isocyanurate triacrylate; ethoxylated₂₀ trimethylolpropane triacrylate; pentaerythritol triacrylate; ethoxylated₃ trimethylolpropane triacrylate; propoxylated₃ trimethylolpropane triacrylate; ethoxylated₆ trimethylolpropane triacrylate; propoxylated₆ trimethylolpropane triacrylate; ethoxylated₉ trimethylolpropane triacrylate; alkoxylated trifunctional acrylate esters; trifunctional methacrylate esters; trifunctional acrylate esters; propoxylated₃ glyceryl triacrylate; propoxylated_{5.5} glyceryl triacrylate; ethoxylated₁₅ trimethylolpropane triacrylate; trifunctional phosphoric acid esters; trifunctional acrylic acid esters; pentaerythritol tetraacrylate; di-trimethylolpropane tetraacrylate; ethoxylated₄ pentaerythritol tetraacrylate; pentaerythrilol polyoxyethylene tetraacrylate; dipentaerythritol pentaacrylate; and pentaacrylate esters.

The curable compositions of the present invention may comprise one or more (meth)acrylate-functionalized monomers containing a single acrylate or methacrylate functional group per molecule (referred to herein as “mono(meth)acrylate-functionalized compounds”), in addition to the glycerol carbonate methacrylate. Any of such compounds known in the art may be used.

Examples of suitable mono(meth)acrylate-functionalized monomers include, but are not limited to, mono-(meth)acrylate esters of aliphatic alcohols (wherein the aliphatic alcohol may be straight chain, branched or alicyclic and may be a mono-alcohol, a di-alcohol or a polyalcohol, provided only one hydroxyl group is esterified with (meth)acrylic acid); mono-(meth)acrylate esters of aromatic alcohols (such as phenols, including alkylated phenols); mono-(meth)acrylate esters of alkylaryl alcohols (such as benzyl alcohol); mono-(meth)acrylate esters of oligomeric and polymeric glycols such as diethylene glycol, triethylene glycol, dipropylene glycol, tripropylene glycol, polyethylene glycol, and polypropylene glycol); mono-(meth)acrylate esters of monoalkyl ethers of glycols and oligoglycols; mono-(meth)acrylate esters of alkoxylated (e.g., ethoxylated and/or propoxylated) aliphatic alcohols (wherein the aliphatic alcohol may be straight chain, branched or alicyclic and may be a mono-alcohol, a di-alcohol or a polyalcohol, provided only one hydroxyl group of the alkoxylated aliphatic alcohol is esterified with (meth)acrylic acid); mono-(meth)acrylate esters of alkoxylated (e.g., ethoxylated and/or propoxylated) aromatic alcohols (such as alkoxylated phenols); caprolactone mono(meth)acrylates; and the like.

The following compounds are specific examples of mono(meth)acrylate-functionalized monomers suitable for use in the curable compositions of the present invention: methyl (meth)acrylate; ethyl (meth)acrylate; n-propyl (meth)acrylate; n-butyl (meth)acrylate; isobutyl (meth)acrylate; n-hexyl (meth)acrylate; 2-ethylhexyl (meth)acrylate; n-octyl (meth)acrylate; isooctyl (meth)acrylate; n-decyl (meth)acrylate; n-dodecyl (meth)acrylate; tridecyl (meth)acrylate; tetradecyl (meth)acrylate; hexadecyl (meth)acrylate; 2-hydroxyethyl (meth)acrylate; 2- and 3-hydroxypropyl (meth)acrylate; 2-methoxyethyl (meth)acrylate; 2-ethoxyethyl (meth)acrylate; 2- and 3-ethoxypropyl (meth)acrylate; tetrahydrofurfuryl (meth)acrylate; alkoxylated tetrahydrofurfuryl (meth)acrylate; isobornyl (meth)acrylate; 2-(2-ethoxyethoxy)ethyl (meth)acrylate; cyclohexyl (meth)acrylate; glycidyl (meth)acrylate; isodecyl (meth)acrylate: 2-phenoxyethyl (meth)acrylate: lauryl (meth)acrylate; 2-phenoxyethyl (meth)acrylate; alkoxylated phenol (meth)acrylates; alkoxylated nonylphenol (meth)acrylates; cyclic trimethylolpropane formal (meth)acrylate; trimethylcyclohexanol (meth)acrylate; diethylene glycol monomethyl ether (meth)acrylate; diethylene glycol monoethyl ether (meth)acrylate; diethylene glycol monobutyl ether (meth)acrylate; triethylene glycol monoethyl ether (meth)acrylate; ethoxylated lauryl (meth)acrylate; methoxy polyethylene glycol (meth)acrylates; hydroxyl ethyl-butyl urethane (meth)acrylates; 3-(2-hydroxyalkyl)oxazolidinone (meth)acrylates; and combinations thereof.

Other types of actinic radiation-curable monomers that could be used in the curable compositions of the present invention include, but are not limited to, cyanoacrylates, vinyl esters, 1,1-diester-1-alkenes, 1,1-diketo-1-alkenes, 1-ester-1-keto-1-alkenes and itaconates, including methylene malonates and/or methylene beta-diketones.

### Stabilizer

Generally speaking, it will be desirable to include one or more stabilizers in the curable compositions of the present invention in order to provide adequate storage stability and shelf life. Advantageously, one or more such stabilizers are present at each stage of the method used to prepare the curable composition, to protect against unwanted reactions during processing of the (meth)acrylate functional groups of components of the curable composition. As used herein, the term “stabilizer” means a compound or substance which retards or prevents reaction or curing of actinically-curable functional groups present in a composition in the absence of actinic radiation. However, it will be advantageous to select an amount and type of stabilizer such that the composition remains capable of being cured when exposed to actinic radiation (that is, the stabilizer does not prevent radiation curing of the composition). Typically, effective stabilizers for purposes of the present invention will be classified as free radical stabilizers (i.e., stabilizers which function by inhibiting free radical reactions).

Any of the stabilizers known in the art related to (meth)acrylate-functionalized compounds may be utilized in the present invention. Quinones represent a particularly preferred type of stabilizer which can be employed in the context of the present invention. As used herein, the term “quinone” includes both quinones and hydroquinones as well as ethers thereof such as monoalkyl, monoaryl, monoaralkyl and bis(hydroxyalkyl) ethers of hydroquinones. Hydroquinone monomethyl ether is an example of a suitable stabilizer which can be utilized.

The concentration of stabilizer in the curable composition will vary depending upon the particular stabilizer or combination of stabilizers selected for use and also on the degree of stabilization desired and the susceptibility of components in the curable compositions towards degradation in the absence of stabilizer. Typically, however, the curable composition is formulated to comprise from 5 to 5000 ppm stabilizer. According to certain embodiments of the invention, the reaction mixture during each stage of the method employed to make the curable composition contains at least some stabilizer, e.g., at least 10 ppm stabilizer.

### Photoinitiator

In certain embodiments of the invention, the curable compositions described herein include at least one photoinitiator and are curable with radiant energy. A photoinitiator may be considered any type of substance that, upon exposure to radiation (e.g., actinic radiation), forms species that initiate the reaction and curing of polymerizing organic substances present in the curable composition. Suitable photoinitiators include both free radical photoinitiators as well as cationic photoinitiators and combinations thereof.

Free radical polymerization initiators are substances that form free radicals when irradiated. The use of free radical photoinitiators is especially preferred. Non-limiting types of free radical photoinitiators suitable for use in the curable compositions of the present invention include, for example, benzoins, benzoin ethers, acetophenones, benzyl, benzyl ketals, anthraquinones, phosphine oxides, α-hydroxyketones, phenylglyoxylates, aminoketones, benzophenones, thioxanthones, xanthones, acridine derivatives, phenazene derivatives, quinoxaline derivatives and triazine compounds.

The amount of photoinitiator may be varied as may be appropriate depending upon the photoinitiator(s) selected, the amounts and types of polymerizable species present in the curable composition, the radiation source and the radiation conditions used, among other factors. Typically, however, the amount of photoinitiator may be from 0.05% to 5%, preferably 0.1% to 2% by weight, based on the total weight of the curable composition.

### Other Additives

The curable compositions of the present invention may optionally contain one or more additives instead of or in addition to the above-mentioned ingredients. Such additives include, but are not limited to, antioxidants/photostabilizers, light blockers/absorbers, polymerization inhibitors, foam inhibitors, flow or leveling agents, colorants, pigments, dispersants (wetting agents, surfactants), slip additives, fillers, chain transfer agents, thixotropic agents, matting agents, impact modifiers, waxes or other various additives, including any of the additives conventionally utilized in the coating, sealant, adhesive, molding, additive manufacturing (e.g., 3D printing) or ink arts.

The curable compositions of the present invention may comprise one or more light blockers (sometimes referred to in the art as absorbers), particularly where the curable composition is to be used as a resin in a three-dimensional printing method involving photocuring of the curable composition. The light blocker(s) may be any such substances known in the three-dimensional printing art, including for example non-reactive pigments and dyes. The light blocker may be a visible light blocker or a UV light blocker, for example. Examples of suitable light blockers include, but are not limited to, titanium dioxide, carbon black and organic ultraviolet light absorbers such as hydroxybenzophenone, hydroxyphenylbenzotriazole, oxanilide, benzophenone, thioxanthone, hydroxyphenyltriazine, Sudan I, bromothymol blue, 2,2′-(2,5-thiophenediyl)bis(5-tert-butylbenzoxazole) (sold under the brand name “Benetex OB Plus”) and benzotriazole ultraviolet light absorbers.

The amount of light blocker may be varied as may be desired or appropriate for particular applications. Generally speaking, if the curable composition contains light blocker, it is present in a concentration of from 0.001 to 10% by weight based on the weight of the curable composition.

Advantageously, the curable compositions of the present invention may be formulated to be solvent-free, i.e., free of any non-reactive volatile substances (substances having a boiling point at atmospheric pressure of 150° C. or less). For example, the curable compositions of the present invention may contain little or no non-reactive solvent, e.g., less than 10% or less than 5% or less than 1% or even 0% non-reactive solvent, based on the total weight of the curable composition. As used herein, the term non-reactive solvent means a solvent that does not react when exposed to the actinic radiation used to cure the curable compositions described herein.

According to other advantageous embodiments of the invention, the curable composition is formulated to be useable as a one component or one part system. That is, the curable composition is cured directly and is not combined with another component or second part (such as an amine monomer, as defined in U.S. Pat. Application Publication No. 2017/0260418 A1) prior to being cured.

### Uses for Curable Compositions

As previously mentioned, curable compositions in accordance with the present invention may contain one or more photoinitiators and may be photocurable. In certain other embodiments of the invention, the curable compositions described herein do not include any initiator and are curable (at least in part) with electron beam energy. In other embodiments, the curable compositions described herein include at least one free radical initiator that decomposes when heated or in the presence of an accelerator and are curable chemically (i.e., without having to expose the curable composition to radiation). The at least one free radical initiator that decomposes when heated or in the presence of an accelerator may, for example, comprise a peroxide or azo compound. Suitable peroxides for this purpose may include any compound, in particular any organic compound, that contains at least one peroxy (—O—O—) moiety, such as, for example, dialkyl, diaryl and aryl/alkyl peroxides, hydroperoxides, percarbonates, peresters, peracids, acyl peroxides and the like. The at least one accelerator may comprise, for example, at least one tertiary amine and/or one or more other reducing agents based on metal-containing salts (such as, for example, carboxylate salts of transition metals such as iron, cobalt, manganese, vanadium and the like and combinations thereof). The accelerator(s) may be selected so as to promote the decomposition of the free radical initiator at room or ambient temperature to generate active free radical species, such that curing of the curable composition is achieved without having to heat or bake the curable composition. In other embodiments, no accelerator is present and the curable composition is heated to a temperature effective to cause decomposition of the free radical initiator and to generate free radical species which initiate curing of the polymerizable compound(s) present in the curable composition.

Advantageously, the curable compositions of the present invention may be formulated to be solvent-free, i.e., free of any non-reactive volatile substances (substances having a boiling point at atmospheric pressure of 150° C. or less). For example, the curable compositions of the present invention may contain little or no non-reactive solvent, e.g., less than 10% or less than 5% or less than 1% or even 0% non-reactive solvent, based on the total weight of the curable composition.

In preferred embodiments of the invention, the curable composition is a liquid at 25° C. In various embodiments of the invention, the curable compositions described herein are formulated to have a viscosity of less than 10,000 mPa·s (cP), or less than 5000 mPa·s (cP), or less than 4000 mPa·s (cP), or less than 3000 mPa·s (cP), or less than 2500 mPa·s (cP), or less than 2000 mPa·s (cP), or less than 1500 mPa·s (cP), or less than 1000 mPa·s (cP) or even less than 500 mPa·s (cP) as measured at 25° C. using a Brookfield viscometer, model DY-II, using a 27 spindle (with the spindle speed varying typically between 20 and 200 rpm, depending on viscosity). In advantageous embodiments of the invention, the viscosity of the curable composition is from 200 to 5000 mPa·s (cP), or from 200 to 2000 mPa·s (cP), or from 200 to 1500 mPa·s (cP), or from 200 to 1000 mPa·s (cP) at 25° C. Relatively high viscosities can provide satisfactory performance in applications where the curable composition is heated above 25° C., such as in three-dimensional printing operations or the like which employ machines having heated resin vats.

The curable compositions described herein may be compositions that are to be subjected to curing by means of free radical polymerization, cationic polymerization or other types of polymerization. In particular embodiments, the curable compositions are photocured (i.e., cured by exposure to actinic radiation such as light, in particular visible or UV light). End use applications for the curable compositions include, but are not limited to, inks, coatings, adhesives, additive manufacturing resins (such as 3D printing resins), molding resins, sealants, composites, antistatic layers, electronic applications, recyclable materials, smart materials capable of detecting and responding to stimuli, packaging materials, personal care articles, articles for use in agriculture, water or food processing, or animal husbandry, and biomedical materials. The curable compositions of the invention thus find utility in the production of biocompatible articles. Such articles may, for example, exhibit high biocompatibility, low cytotoxicity and/or low extractables.

Cured compositions prepared from curable compositions as described herein may be used, for example, in three-dimensional articles (wherein the three-dimensional article may consist essentially of or consist of the cured composition), coated articles (wherein a substrate is coated with one or more layers of the cured composition, including encapsulated articles in which a substrate is completely encased by the cured composition), laminated or adhered articles (wherein a first component of the article is laminated or adhered to a second component by means of the cured composition), composite articles or printed articles (wherein graphics or the like are imprinted on a substrate, such as a paper, plastic or M-containing substrate, using the cured composition).

Curing of the curable compositions in accordance with the present invention may be carried out by any suitable method, such as free radical and/or cationic polymerization. One or more initiators, such as a free radical initiator (e.g., photoinitiator, peroxide initiator) may be present in the curable composition. Prior to curing, the curable composition may be applied to a substrate surface in any known conventional manner, for example, by spraying, knife coating, roller coating, casting, drum coating, dipping, and the like and combinations thereof. Indirect application using a transfer process may also be used. A substrate may be any commercially relevant substrate, such as a high surface energy substrate or a low surface energy substrate, such as a metal substrate or plastic substrate, respectively. The substrates may comprise metal, paper, cardboard, glass, thermoplastics such as polyolefins, polycarbonate, acrylonitrile butadiene styrene (ABS), and blends thereof, composites, wood, leather and combinations thereof. When used as an adhesive, the curable composition may be placed between two substrates and then cured, the cured composition thereby bonding the substrates together to provide an adhered article. Curable compositions in accordance with the present invention may also be formed or cured in a bulk manner (e.g., the curable composition may be cast into a suitable mold and then cured).

Curing may be accelerated or facilitated by supplying energy to the curable composition, such as by heating the curable composition and/or by exposing the curable composition to a radiation source, such as visible or UV light, infrared radiation, and/or electron beam radiation. Thus, the cured composition may be deemed the reaction product of the curable composition, formed by curing. A curable composition may be partially cured by exposure to actinic radiation, with further curing being achieved by heating the partially cured article. For example, an article formed from the curable composition (e.g., a 3D printed article) may be heated at a temperature of from 40° C. to 120° C. for a period of time of from 5 minutes to 12 hours.

A plurality of layers of a curable composition in accordance with the present invention may be applied to a substrate surface; the plurality of layers may be simultaneously cured (by exposure to a single dose of radiation, for example) or each layer may be successively cured before application of an additional layer of the curable composition.

The curable compositions which are described herein can be used as resins in three-dimensional printing applications. Three-dimensional (3D) printing (also referred to as additive manufacturing) is a process in which a 3D digital model is manufactured by the accretion of construction material. The 3D printed object is created by utilizing the computer-aided design (CAD) data of an object through sequential construction of two dimensional (2D) layers or slices that correspond to cross-sections of 3D objects. Stereolithography (SL) is one type of additive manufacturing where a liquid resin is hardened by selective exposure to a radiation to form each 2D layer. The radiation can be in the form of electromagnetic waves or an electron beam. The most commonly applied energy source is ultraviolet, visible or infrared radiation.

Sterolithography and other photocurable 3D printing methods typically apply low intensity light sources to radiate each layer of a photocurable resin to form the desired article. As a result, photocurable resin polymerization kinetics and the flexural strength (green strength) of the printed article are important criteria if a particular photocurable resin will sufficiently polymerize (cure) when irradiated and have sufficient engineered material flexural strength to retain its integrity through the 3D printing process. As previously mentioned, acrylates and methacrylates typically have different reactivities that can be explained by steric hindrance and charge induction by the methyl group of a methacrylate in the alpha position of the double bond that consequently encumbers the polymerization rate. Glycerol carbonate methacrylate, however, has been reported to have a reactivity rate 1.7 times higher than methyl methacrylate and 7 times higher than glycidyl methacrylate under similar polymerization conditions (Camara et al., European Polymer Journal 61 (2014) 133-144).

The kinetics of radiation curing in a copolymerization of an acrylate having a certain structure with its corresponding methacrylate generally do not produce a kinetic rate which is the average of the kinetic rates of the individual acrylate and methacrylate, as the kinetics of the methacrylate typically prevail. However, glycerol carbonate methacrylate has been found to be atypical in its reactivity and the present inventors have discovered that glycerol carbonate methacrylate (GCMA) may be effectively used in curable compositions based on methacrylate-functionalized compounds to increase their radiation-induced cure speeds and improve the flexural strength of the cured products derived therefrom, thus making such GCMA-modified formulations particularly useful in 3D printing applications. That is, GCMA may be used in 3D printing resin compositions containing one or more other methacrylates to improve the degree of conversion achieved within a predetermined period of time, despite the presence of slow reacting methacrylates.

The inventive curable compositions described herein thus are especially useful as 3D printing resin formulations, that is, compositions intended for use in manufacturing three-dimensional articles using 3D printing techniques. Such three-dimensional articles may be free-standing/self-supporting and may consist essentially of or consist of a composition in accordance with the present invention that has been cured. The three-dimensional article may also be a composite, comprising at least one component consisting essentially of or consisting of a cured composition as previously mentioned as well as at least one additional component comprised of one or more materials other than such a cured composition (for example, a metal component or a thermoplastic component). The curable compositions of the present invention are particularly useful in digital light printing (DLP), although other types of three-dimensional (3D) printing methods may also be practiced using the inventive curable compositions (e.g., SLA, inkjet, multi jet printing, piezoelectric printing, actinically-cured extrusion, and gel deposition printing). The curable compositions of the present invention may be used in a three-dimensional printing operation together with another material which functions as a scaffold or support for the article formed from the curable composition of the present invention.

Thus, the curable compositions of the present invention are useful in the practice of various types of three-dimensional fabrication or printing techniques, including methods in which construction of a three-dimensional object is performed in a step-wise or layer-by-layer manner. In such methods, layer formation may be performed by solidification (curing) of the curable composition under the action of exposure to radiation, such as visible, UV or other actinic irradiation. For example, new layers may be formed at the top surface of the growing object or at the bottom surface of the growing object. The curable compositions of the present invention may also be advantageously employed in methods for the production of three-dimensional objects by additive manufacturing wherein the method is carried out continuously. For example, the object may be produced from a liquid interface. Suitable methods of this type are sometimes referred to in the art as “continuous liquid interface (or interphase) product (or printing)” (“CLIP”) methods. Such methods are described, for example, in WO 2014/126830; WO 2014/126834; WO 2014/126837; and Tumbleston et al.,

“Continuous Liquid Interface Production of 3D Objects,” Science Vol. 347, Issue 6228, pp. 1349-1352 (Mar. 20, 2015), the entire disclosure of which is incorporated herein by reference in its entirety for all purposes.

When stereolithography is conducted above an oxygen-permeable build window, the production of an article using a curable composition in accordance with the present invention may be enabled in a CLIP procedure by creating an oxygen-containing “dead zone” which is a thin uncured layer of the curable composition between the window and the surface of the cured article as it is being produced. In such a process, a curable composition is used in which curing (polymerization) is inhibited by the presence of molecular oxygen; such inhibition is typically observed, for example, in curable compositions which are capable of being cured by free radical mechanisms. The dead zone thickness which is desired may be maintained by selecting various control parameters such as photon flux and the optical and curing properties of the curable composition. The CLIP process proceeds by projecting a continuous sequence of actinic radiation (e.g., UV) images (which may be generated by a digital light-processing imaging unit, for example) through an oxygen-permeable, actinic radiation- (e.g., UV-) transparent window below a bath of the curable composition maintained in liquid form. A liquid interface below the advancing (growing) article is maintained by the dead zone created above the window. The curing article is continuously drawn out of the curable composition bath above the dead zone, which may be replenished by feeding into the bath additional quantities of the curable composition to compensate for the amounts of curable composition being cured and incorporated into the growing article.

In view of the relatively low toxicity of the glycerol carbonate methacrylate component, the curable compositions of the present invention are particularly useful in the fabrication of articles intended for biomedical or skin contact applications, such as applications in the fields of dentistry, prosthetics, implantable devices, surgical instruments, and tissue and organ replacement. Thus, in some embodiments, the article prepared from the curable composition is for use in a context that places the article in direct or close contact with an organism (e.g., an animal or human) at risk from toxic effects or with substances to be consumed by such an organism (e.g., food, drinking water, pharmaceuticals, personal care products), such as medical and dental articles, personal care articles, toys, packaging for food, beverage and personal care products, and articles used in the fields of food, beverage and water processing, agriculture and animal husbandry. For such end-uses, the other components of the curable composition should of course also be selected to have relatively low toxicity (including little to no tendency to provoke allergic, inflammatory, or sensitization responses in an organism, such as a human being).

### Aspects of the Invention

Illustrative, non-limiting embodiments of the present invention may be summarized as follows:

Aspect 1: A method of making a glycerol carbonate (meth)acrylate, wherein the method comprises reacting a glycerol mono(meth)acrylate and a carbonate selected from the group consisting of dialkyl carbonates and cyclic alkylene carbonates in the presence of a catalyst.

Aspect 2: The method of Aspect 1, wherein the catalyst is selected from Lewis acids or Lewis bases.

Aspect 3: The method of Aspect 1, wherein the catalyst is a Bronsted basic catalyst.

Aspect 4: The method of any one of Aspects 1 to 3, wherein the catalyst is selected from the group consisting of alkali metal hydroxides and alkali metal alkoxides.

Aspect 5: The method of any one of Aspects 1 to 4, wherein the carbonate is selected from the group consisting of dimethyl carbonate, diethyl carbonate, dipropylcarbonates, ethylene carbonate and propylene carbonate.

Aspect 6: The method of any one of Aspects 1 to 5, wherein the glycerol mono(meth)acrylate and carbonate are reacted at a temperature of 40 to 160° C.

Aspect 7: The method of any one of Aspects 1 to 6, wherein the reacting of the glycerol mono(meth)acrylate and the carbonate takes place in a liquid phase.

Aspect 8: The method of Aspect 7, where a co-product alcohol is formed during the reacting.

Aspect 9: The method of Aspect 8, wherein the co-product alcohol is removed from the liquid phase during the reacting.

Aspect 10: The method of any one of Aspects 1 to 9, wherein the carbonate and the glycerol mono(meth)acrylate are reacted in a molar ratio of carbonate : glycerol mono(meth)acrylate of from 1:1 to 3:1.

Aspect 11: The method of any one of Aspects 1 to 10, wherein the reacting is carried out in the presence of a polymerization inhibitor.

Aspect 12: A curable composition, comprising glycerol carbonate methacrylate and at least one actinic radiation-curable oligomer (according to one aspect, the curable composition does not comprise any actinic radiation-curable oligomer containing amino groups).

Aspect 13: The curable composition of Aspect 12, wherein the at least one actinic radiation-curable oligomer comprises at least one (meth)acrylate-functionalized oligomer selected from the group consisting of (meth)acrylate-functionalized urethane oligomers, (meth)acrylate-functionalized epoxy oligomers, (meth)acrylate-functionalized polyether oligomers, (meth)acrylate-functionalized polydiene oligomers, (meth)acrylate-functionalized polycarbonate oligomers, and (meth)acrylate-functionalized polyester oligomers.

Aspect 14: The curable composition of Aspect 12 or 13, wherein the at least one (meth)acrylated oligomer has a viscosity at 25° C. in neat form of at least 10,000 cPS and the glycerol carbonate methacrylate is present in an amount effective to provide the curable composition with a viscosity at 25° C. of less than 10,000 cPs.

Aspect 15: The curable composition of any one of Aspects 12 to 14, wherein upon curing the curable composition provides a cured polymeric matrix having both a higher tensile modulus, as measured by ASTM D638-14 (Type IV), and a higher Notched Izod impact resistance, as measured by ASTM D256-10(2018), than a cured polymeric matrix obtained by curing an analogous curable composition having an identical composition except for the substitution of ethoxylated₂ bisphenol A diacrylate monomer for the glycerol carbonate methacrylate.

Aspect 16: The curable composition of any one of Aspects 12 to 15, additionally comprising at least one actinic radiation-curable monomer other than glycerol carbonate methacrylate.

Aspect 17: The curable composition of any one of Aspects 12 to 16, additionally comprising at least one actinic radiation-curable monomer other than glycerol carbonate methacrylate selected from the group consisting of cyanoacrylates, vinyl esters, 1,1-diester-1-alkenes, 1,1-diketo-1-alkenes, 1-ester-1-keto-1-alkenes and itaconates.

Aspect 18: The curable composition of any one of Aspects 12 to 17, additionally comprising at least one methacrylate-functionalized monomer other than glycerol carbonate methacrylate.

Aspect 19: A method of additive manufacturing, wherein the method comprises radiation-curing a curable composition comprised of glycerol carbonate methacrylate and at least one (meth)acrylate functionalized oligomer but no amino-containing compound.

Aspect 20: A method of additive manufacturing comprising radiation-curing a one-part curable composition comprised of glycerol carbonate methacrylate, wherein the one-part curable composition does not comprise any amino-containing compound and is not combined with any amino-containing compound prior to being radiation-cured.

Within this specification, embodiments have been described in a way which enables a clear and concise specification to be written, but it is intended and will be appreciated that embodiments may be variously combined or separated without departing from the invention. For example, it will be appreciated that all preferred features described herein are applicable to all aspects of the invention described herein.

In some embodiments, the invention herein can be construed as excluding any element or process step that does not materially affect the basic and novel characteristics of the invention. Additionally, in some embodiments, the invention can be construed as excluding any element or process step not specified herein.

Although the invention is illustrated and described herein with reference to specific embodiments, the invention is not intended to be limited to the details shown. Rather, various modifications may be made in the details within the scope and range of equivalents of the claims and without departing from the invention.

### EXAMPLES

### Example 1

This Example demonstrates that glycerol carbonate methacrylate (GCMA) is effective for increasing cure speed and improving green (flexural) strength when copolymerized with other methacrylates.

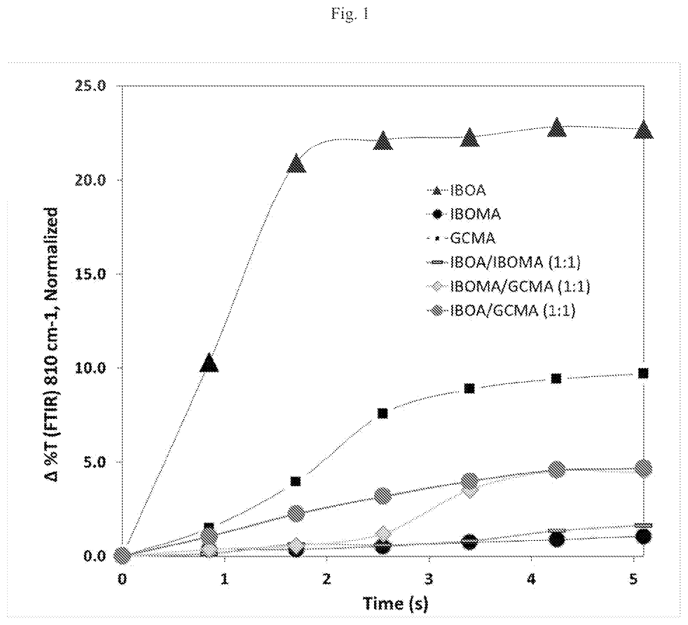

The study below (Table 1; FIG. 1) shows that the change in % Transmittance of the 810 cm⁻¹ peak in the FTIR spectrum is attributed to the C═O in plane and out of plane vibratory motion of (meth)acrylate systems. All samples were prepared with 0.5% Irgacure® 819 photoinitiator. Observing the changes to this peak for isobornyl acrylate (IBOA/sold by Sartomer as SR506) yields a 22.7% normalized % Transmittance (% T_{N}) when irradiated with UV light over the course of 5.1 seconds, with the slowest conversions achieved with isobornyl methacrylate (IBOMA/sold by Sartomer as SR423) displaying 1.0% T_{N} at 5.1 seconds. Neat GCMA provided a slower conversion rate of 9.7% T_{N} than IBOA, but this was faster than all other neat methacrylates evaluated. The combination of IBOA and IBOMA yields only a marginal improvement in conversion (1.6% T_{N}) over neat IBOMA. However, combining GCMA and IBOMA yields a conversion rate of 4.5% T_{N}, a nearly 3-fold improvement in conversion than the addition of the kinetically faster isobornyl acrylate/isobornyl methacrylate system. These results show that the use of GCMA in 3D printed applications in systems containing methacrylates is of immense utility to improve the degree of conversion in the presence of slower reacting methacrylates.

TABLE 1

| | Δ % T (FTIR) 810 | | | IBOA/ | IBOMA/ | IBOA/ |
|---|---|---|---|---|---|---|
| | cm−1, Normalized | | | IBOMA | GCMA | GCMA |
| TIME(s) | IBOA | IBOMA | GCMA | (1:1) | (1:1) | (1:1) |
| 0 | 0.0 | 0.0 | 0.0 | 0.0 | 0.0 | 0.0 |
| 0.85 | 10.3 | 0.3 | 1.5 | 0.1 | 0.3 | 1.0 |
| 1.7 | 20.9 | 0.4 | 3.9 | 0.6 | 0.6 | 2.2 |
| 2.55 | 22.1 | 0.5 | 7.6 | 0.6 | 1.2 | 3.2 |
| 3.4 | 22.3 | 0.7 | 8.9 | 0.8 | 3.5 | 4.0 |
| 4.25 | 22.8 | 0.9 | 9.4 | 1.3 | 4.5 | 4.6 |
| 5.1 | 22.7 | 1.0 | 9.7 | 1.6 | 4.5 | 4.7 |

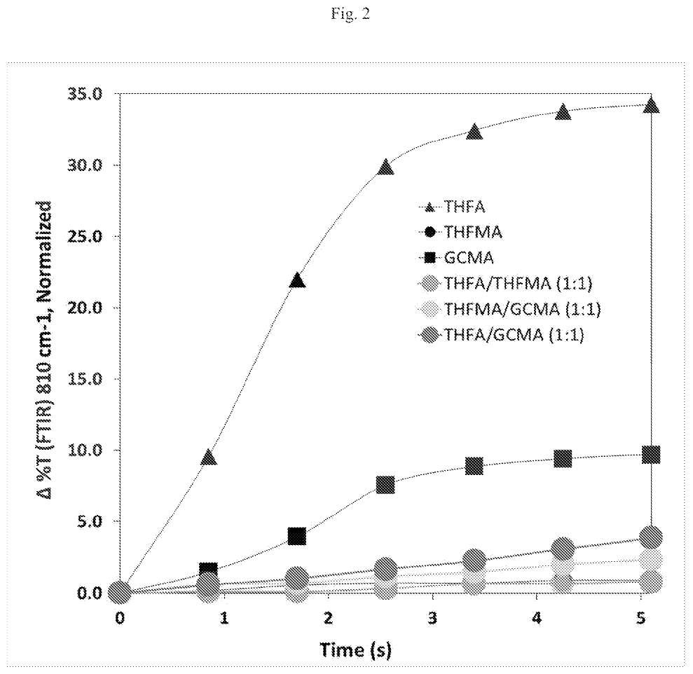

A similar study was undertaken involving tetrahydrofuryl acrylate (THFA), tetrahydrofuryl methacrylate (THFMA) and GCMA. The results obtained are set forth in Table 2 and FIG. 2. Here, THFA displays the greatest conversion at 34.3% T_{N} when irradiated with UV light over the course of 5.1 seconds, while THFMA exhibited a reduced conversion of 0.9% T_{N}. GCMA once again displayed a % T_{N} of 9.7, which is an order of magnitude greater conversion than THFMA. Kinetic polymerization rates of 1:1 blends of THFA and THFMA are predominated by the THFMA with a 0.8% T_{N}. Combinations of THFMA and GCMA yield a 2.4% T_{N}, a 3-fold improvement over neat THFMA or blends of THFMA. As evidenced in this parallel study, GCMA expedites the kinetics of slower reacting methacrylates.

TABLE 2

| | | | | THFA/ | THFMA/ | THFA/ |
|---|---|---|---|---|---|---|
| | | | | THFMA | GCMA | GCMA |
| | THFMA | THFA | GCMA | (1:1) | (1:1) | (1:1) |
| 0 | 0 | 0 | 0 | 0 | 0 | 0 |
| 0.85 | 0.2 | 9.6 | 1.5 | 0.1 | 0.6 | 0.6 |
| 1.7 | 0.6 | 22.0 | 3.9 | 0.1 | 0.7 | 1.0 |
| 2.55 | 0.7 | 29.9 | 7.6 | 0.3 | 1.2 | 1.7 |
| 3.4 | 0.7 | 32.4 | 8.9 | 0.6 | 1.5 | 2.2 |
| 4.25 | 0.9 | 33.8 | 9.4 | 0.7 | 2.0 | 3.1 |
| 5.1 | 0.9 | 34.3 | 9.7 | 0.8 | 2.4 | 3.9 |

Materials and Methods:

Formulations were prepared using Sartomer's SR506, SR423, SR203, and SR285 products and glycerol carbonate methacrylate (GCMA) with 0.5% Irgacure® 819 (BASF) and mixed until homogenous at room temperature. Formulations were photocured on a Nicolet IS50 FTIR spectrophotometer with an attenuated total reflectance (ATR) accessory fitted with a Dymax BlueWave LED Prime UVA (Aₘₐₓ=385 nm). Photopolymerization was initiated concurrently with kinetic data collection at fixed 0.85 s intervals at 810 cm⁻¹.

Monomers (all products of Sartomer):

SR506—Isobornyl Acrylate (IBOA)

SR423—Isobornyl Methacrylate (IBOMA)

SR203—Tetrahydrofurfuryl Methacrylate (THFMA)

SR285—Tetrahydrofurfuryl Acrylate (THFA)

### Example 2

UV-curable formulations were prepared by mixing resin components and photoinitiator in glass sample jars and placing the jars on rollers in a 60° C. oven overnight to ensure adequate mixing. After mixing, the formulations were poured into silicone molds and cured by passing the mold under a UV light source (e.g. 395 nm LED at a belt speed of 50 feet per minute), forming solid test specimens for tensile testing, dynamic mechanical analysis (DMA), and notched Izod impact resistance.

ASTM D638 (Type IV) was used to obtain tensile data, ASTM D256 was used for Notched Izod impact resistance, and a TA Q800 DMA was used for glass transition temperature (defined as the tan-δ peak) and the glass transition onset temperature (defined as the G″ peak). A temperature-controlled cone-and-plate rheometer was used to obtain viscosity data.

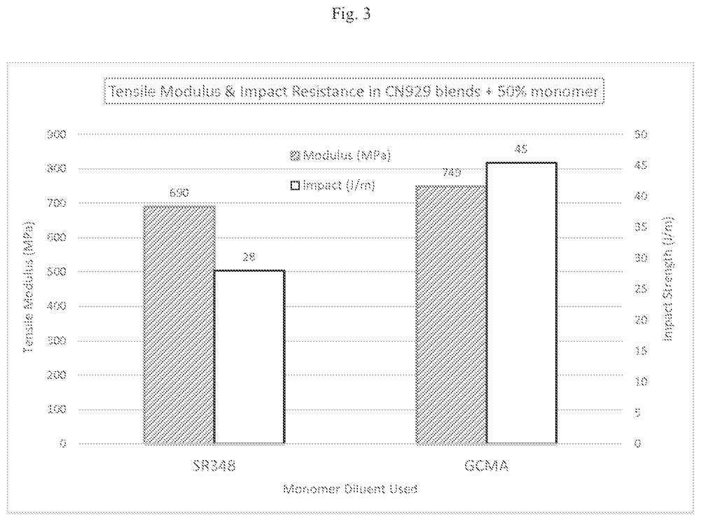

FIG. 3 compares the tensile and impact properties of cured specimens of two 3D-printable compositions: a blend of 50 wt % CN929 (a urethane acrylate oligomer sold by Sartomer) and 50 wt % SR348 (ethoxylated₂ bisphenol A diacrylate monomer sold by Sartomer, containing about 2 moles oxyethylene per mole), and a similar composition with 50% GCMA replacing the ethoxylated BPA diacrylate monomer. In many (meth)acrylate-based 3D printing compositions, an increase in modulus is usually accompanied by a decrease in impact resistance. In this case, switching to GCMA achieves an increase in both properties simultaneously.

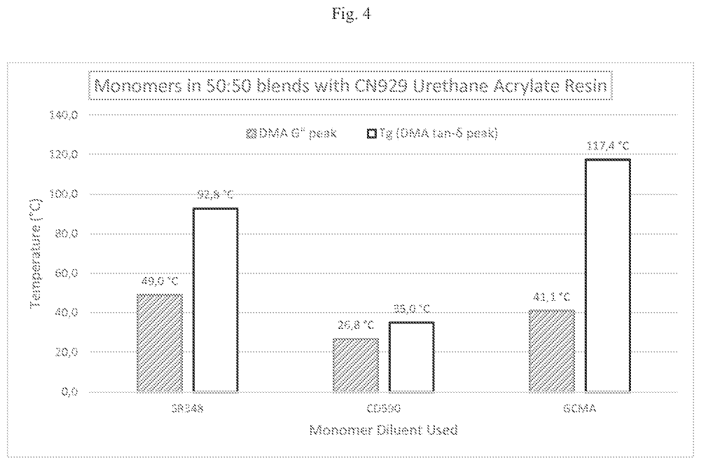

FIG. 4 compares the thermal properties of three 3D-printable compositions containing 50 wt % monomer and 50 wt % CN929 (a urethane acrylate oligomer sold by Sartomer). Typically, switching from a difunctional (meth)acrylate monomer to a similar monofunctional (meth)acrylate monomer is accompanied by a decrease in the glass transition temperature, such as interchanging SR348 for CD590 (a monofunctional aromatic acrylate monomer sold by Sartomer) in the above example. However, interchanging difunctional SR348 to monofunctional GCMA achieves a 25° C. increase in glass transition temperature, enabling 3D-printed parts from GCMA-containing 3D printable compositions to retain their mechanical properties across a larger range of elevated temperatures.

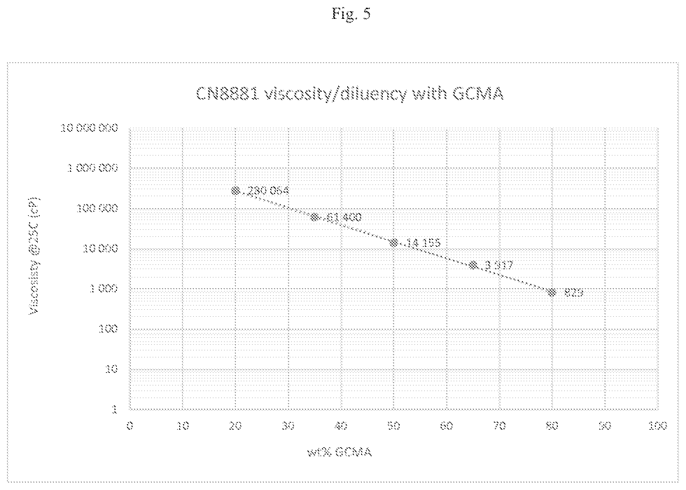

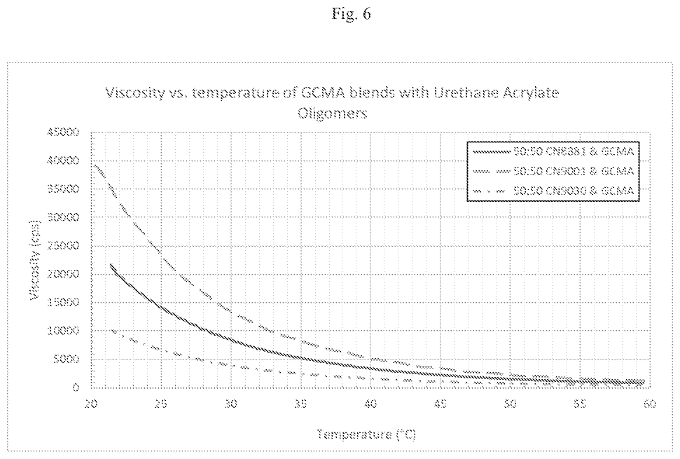

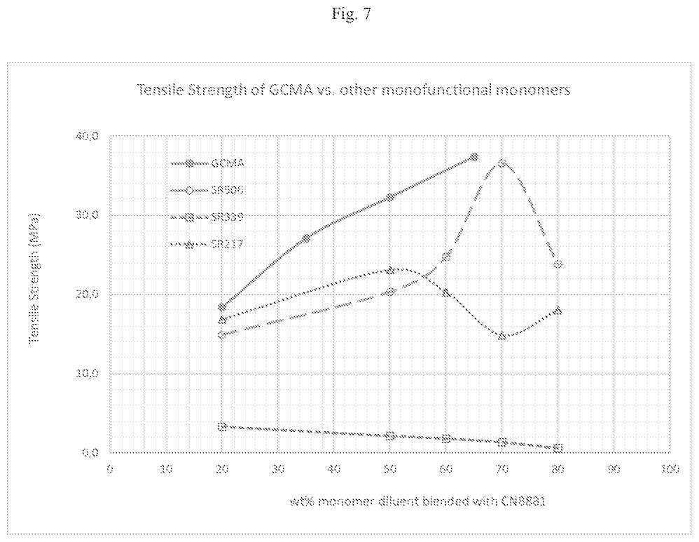

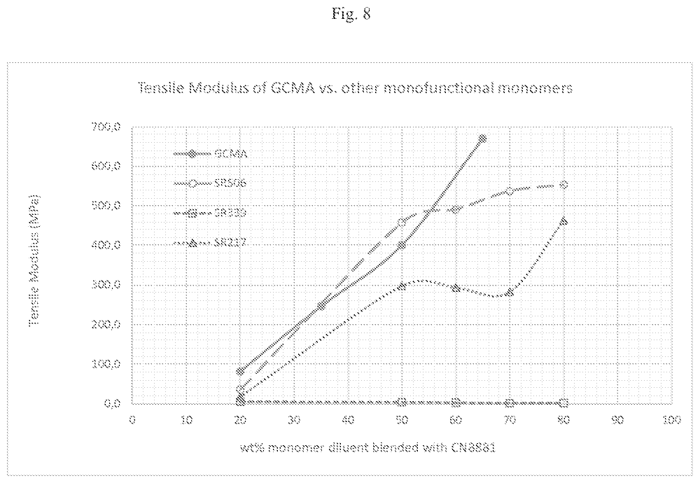

FIG. 5 shows how the room temperature viscosity of CN2881 (a highly branched multifunctional polyester acrylate oligomer sold by Sartomer) may be reduced by blending with increased amounts of GCMA. FIG. 6 provides viscosity vs. temperature curves for 50:50 blends of various (meth)acrylate-functionalized oligomers with GCMA. CN8881 is a difunctional urethane acrylate sold by Sartomer; CN9001 is an aliphatic urethane acrylate oligomer sold by Sartomer; and CN9030 is a difunctional aliphatic urethane acrylate oligomer sold by Sartomer. Tables 3 and 4 and FIGS. 7 and 8 compare the tensile strength and tensile modulus of cured samples prepared from blends of various types of (meth)acrylate-functionalized monomers (including GCMA) with (meth)acrylate-functionalized oligomer (CN8881—difunctional urethane acrylate sold by Sartomer), showing the results obtained using differing amounts of monomer.

Monomers (all products of Sartomer):

SR506—Isobornyl Acrylate

SR339—2-Phenoxyethyl Acrylate

SR217-4-tert-Butylcyclohexyl Acrylate

TABLE 3

| | 1 | 2 | 3 | 4 | 5 | 6 | 7 | 8 | 9 | 10 |
|---|---|---|---|---|---|---|---|---|---|---|
| CN8881 | 30 | 30 | 40 | 30 | 20 | 80 | 50 | 40 | 30 | 20 |
| SR506 | 20 | 50 | 50 | 70 | 80 | | | | | |
| SR339 | | | | | | 20 | 50 | 50 | 70 | 80 |
| SR217 | | | | | | | | | | |
| GCMA | | | | | | | | | | |
| Irgacure 819 | 0.5 | 0.5 | 0.5 | 0.5 | 0.5 | 0.5 | 0.5 | 0.5 | 0.5 | 0.5 |
| Viscosity @ 25° C. (cP) | 30,000 | 2,715 | 795 | 265 | 75 | 50,000 | 4,338 | 535 | 242 | 55 |
| ASTM D638 Tensile | 24.9 | 20.4 | 24.5 | 35.5 | 23.8 | 3.3 | 2.1 | 1.8 | 1.4 | 0.5 |
| Strength (MPa) | | | | | | | | | | |
| ASTM D638 | 159 | 51 | 44 | 19 | 5 | 23 | 97 | 123 | 150 | 131 |
| Elongation (%) | | | | | | | | | | |
| ASTM D638 Tensile | 3.5 | 45.9 | 49.1 | 53.8 | 55.4 | 5 | 4 | 3 | 2 | 2 |
| Modulus (MPa) | | | | | | | | | | |

TABLE 4

| | 11 | 12 | 13 | 14 | 15 | 16 | 17 | 18 | 19 | 20 |
|---|---|---|---|---|---|---|---|---|---|---|
| CN8881 | 50 | 50 | 40 | 30 | 20 | 80 | 65 | 50 | 35 | 20 |
| SR506 | | | | | | | | | | |
| SR339 | | | | | | | | | | |
| SR217 | 20 | 50 | 50 | 70 | 80 | | | | | |
| GCMA | | | | | | 20 | 35 | 50 | 65 | 80 |
| Irgacure 819 | 0.5 | 0.5 | 0.5 | 0.5 | 0.5 | 0.5 | 0.5 | 0.5 | 0.5 | 0.5 |
| Viscosity @ 25° C. (cP) | 30,000 | 3,800 | 1,700 | 430 | 15.5 | 250,054 | 51,400 | 14,155 | 3,317 | 829 |
| ASTM D638 Tensile | 15.1 | 23.1 | 20.3 | 14.3 | 18.1 | 18.4 | 27.1 | 32.3 | 37.4 | — |
| Strength (MPa) | | | | | | | | | | |
| ASTM D638 | 183 | 205 | 233 | 229 | 8 | 156.2 | 83 | 39.1 | 2.9 | — |
| Elongation (%) | | | | | | | | | | |
| ASTM D638 Tensile | 1.8 | 29.8 | 29.3 | 28.3 | 45.4 | 82.2 | 245.7 | 401.4 | 570.5 | — |
| Modulus (MPa) | | | | | | | | | | |

### Example 3—Synthesis of Glycerol Carbonate Methacrylate

To a 4-neck flask equipped with an overhead stirrer, addition funnel, thermocouple, and air sparge tube was added glycerol monomethacrylate (200 g, 1.0 equiv., 1.25 mol), 4-methoxyphenol (0.3 g, 500 ppm with respect to the quantitative product), and sodium hydroxide (0.54 g 1500 ppm). Diethylcarbonate (162.3 g, 1.1 equiv.) was loaded into the addition funnel and atmospheric air was continuously bubbled throughout the course of the reaction into the flask through the sparge tube. To the flask from the addition funnel was added 24.3 g of diethylcarbonate at room temperature and the flask was heated to 75° C. wherein an exotherm was noted to raise the pot temperature to a reflux temperature of 90° C. The reaction mixture was allowed to cool to 85° C. before an additional aliquot of diethylcarbonate (105 g) was added over the course of 5 minutes. After an additional 30 minutes at 85° C., the remaining diethylcarbonate was added and the reaction mixture was then allowed to stir for an additional 30 minutes, or until conversion exceeded 80% by GC determination. The reaction mixture was cooled to 60° C. and placed under reduced pressure (300 torr) to remove the ethanol byproduct and promote conversion to the product for 1 h. The crude product mixture was then heated to 90° C. under reduced pressure (<30 torr) to remove residual diethylcarbonate and yielded glycerol carbonate methacrylate (227 g, 98% GC) as a colorless oil.

1. A method of making a glycerol carbonate (meth)acrylate, wherein the method comprises reacting a glycerol mono(meth)acrylate and a carbonate selected from the group consisting of dialkyl carbonates and cyclic alkylene carbonates in the presence of a catalyst.

2. The method of claim 1, wherein the catalyst is selected from Lewis acids or Lewis bases.

3. The method of claim 1, wherein the catalyst is a Bronsted basic catalyst.

4. The method of claim 1, wherein the catalyst is selected from the group consisting of alkali metal hydroxides and alkali metal alkoxides.

5. The method of claim 1, wherein the carbonate is selected from the group consisting of dimethyl carbonate, diethyl carbonate, dipropylcarbonates, ethylene carbonate and propylene carbonate.

6. The method of claim 1, wherein the glycerol mono(meth)acrylate and carbonate are reacted at a temperature of 40 to 160° C.

7. The method of claim 1, wherein the reacting of the glycerol mono(meth)acrylate and the carbonate takes place in a liquid phase.

8. The method of claim 7, where a co-product alcohol is formed during the reacting.

9. The method of claim 8, wherein the co-product alcohol is removed from the liquid phase during the reacting.

10. The method of claim 1, wherein the carbonate and the glycerol mono(meth)acrylate are reacted in a molar ratio of carbonate:glycerol mono(meth)acrylate of from 1:1 to 3:1.

11. The method of claim 1, wherein the reacting is carried out in the presence of a polymerization inhibitor.

12. A curable composition, comprising glycerol carbonate methacrylate and at least one actinic radiation-curable oligomer.

13. The curable composition of claim 12, wherein the at least one actinic radiation-curable oligomer comprises at least one (meth)acrylate-functionalized oligomer selected from the group consisting of (meth)acrylate-functionalized urethane oligomers, (meth)acrylate-functionalized epoxy oligomers, (meth)acrylate-functionalized polyether oligomers, (meth)acrylate-functionalized polydiene oligomers, (meth)acrylate-functionalized polycarbonate oligomers, and (meth)acrylate-functionalized polyester oligomers.

14. The curable composition of claim 13, wherein the at least one (meth)acrylated oligomer has a viscosity at 25° C. in neat form of at least 10,000 cPS and the glycerol carbonate methacrylate is present in an amount effective to provide the curable composition with a viscosity at 25° C. of less than 10,000 cPs.

15. The curable composition of claim 12, wherein upon curing the curable composition provides a cured polymeric matrix having both a higher tensile modulus, as measured by ASTM D638-14 (Type IV), and a higher Notched Izod impact resistance, as measured by ASTM D256-10(2018), than a cured polymeric matrix obtained by curing an analogous curable composition having an identical composition except for the substitution of ethoxylated₂ bisphenol A diacrylate monomer for the glycerol carbonate methacrylate.

16. The curable composition of claim 12, additionally comprising at least one actinic radiation-curable monomer other than glycerol carbonate methacrylate.

17. The curable composition of claim 12, additionally comprising at least one actinic radiation-curable monomer other than glycerol carbonate methacrylate selected from the group consisting of cyanoacrylates, vinyl esters, 1,1-diester-1-alkenes, 1,1-diketo-1-alkenes, 1-ester-1-keto-1-alkenes and itaconates.

18. The curable composition of claim 12, additionally comprising at least one methacrylate-functionalized monomer other than glycerol carbonate methacrylate.

19. A method of additive manufacturing, wherein the method comprises radiation-curing a curable composition comprised of glycerol carbonate methacrylate and at least one (meth)acrylate functionalized oligomer but no amino-containing compound.

20. A method of additive manufacturing comprising radiation-curing a one-part curable composition comprised of glycerol carbonate methacrylate, wherein the one-part curable composition does not comprise any amino-containing compound and is not combined with any amino-containing compound prior to being radiation-cured.
